(19) United States
(12) Patent Application Publication
(10) Pub. No.: US 2019/0178821 A1
(43) Pub. Date: Jun. 13, 2019

(54) X-Ray Tomography Inspection Systems and Methods

(71) Applicant: Rapiscan Systems, Inc., Torrance, CA (US)

(72) Inventor: Edward James Morton, Guildford (GB)

(21) Appl. No.: 16/208,193

(22) Filed: Dec. 3, 2018

Related U.S. Application Data
(60) Provisional application No. 62/597,155, filed on Dec. 11, 2017.

(51) Int. Cl. G01N 23/083; A61B 6/03; H01J 35/08; H01J 35/06

(52) U.S. Cl. CPC G01N 23/083; H01J 35/06; H01J 35/08; A61B 6/03

(57) ABSTRACT

An optical assembly for use in an X-ray inspection system. The optical assembly has a light source, a photocathode positioned such that it is in a path of light emitted by the light source, and at least two dynodes. One of the dynodes is positioned to receive electrons emitted by the photocathode and the other dynode is positioned to receive electrons emitted by the first dynode. The light source is preferably one of an LED light source or a LASER light source.

### CROSS-REFERENCE

The present application relies on U.S. Provisional Patent Application No. 62/597,155, entitled “X-Ray Tomography Inspection Systems and Methods” and filed on Dec. 11, 2017, for priority, which is herein incorporated by reference in its entirety.

### FIELD

The present specification relates to X-ray scanning systems. More particularly, the present specification relates to a stationary gantry X-ray inspection system having a plurality of X-ray sources positioned around a volume of inspection such that the sources emit X-ray beams having different beam angles.

### BACKGROUND

X-ray computed tomography (CT) scanners have been used in security screening in airports for several years. A conventional system comprises an X-ray tube that is rotated about an axis with an arcuate X-ray detector which is also rotated, at the same speed, around the same axis. The conveyor belt on which the baggage is carried is placed within a suitable aperture around the central axis of rotation, and moved along the axis as the tube is rotated. A fan beam of X-radiation passes from the source through the object to be inspected and subsequently to the X-ray detector array.

The X-ray detector array records the intensity of X-rays passed through the object to be inspected at several locations along its length. One set of projection data is recorded at each of a number of source angles. From these recorded X-ray intensities, it is possible to form a tomographic (cross-sectional) image, typically by means of a filtered back projection algorithm. Rotational scanning of the X-ray source is applied in order to produce an accurate tomographic image of an object, such as a bag or package, during the longitudinal motion of the conveyor on which the object is carried.

In a conventional X-ray scanner system, an X-ray tube comprises an electron source that is designed to emit electrons towards an anode which is held at high positive potential (typically in the range 15 kV to 450 kV) with respect to the electron source. Electrons emitted at low potential from the electron source are accelerated in the electric field that exists between the electron source (cathode) and the anode. When the accelerated electrons impact on the anode surface, a fraction (typically 1%) of their energy is emitted as X-radiation, the balance resulting in thermal heating of the anode or electrons backscattered from the anode.

Typically an electron source for an X-ray tube comprises a thermionic emitter such as a heated tungsten wire. Electrons in the wire can gain sufficient energy to “boil” off from the surface of the wire into the surrounding vacuum from where they may be extracted into an electric field that exists between the cathode and anode.

Such electron sources are very commonly used, typically in a coiled form, in a wide range of X-ray tubes. Such sources are characterized by an operating temperature upwards of 1500K with a filament resistance of a few ohms when at operating temperature and an operating power dissipation in the range 1 W to 20 W depending on the application in which the tube is designed to be used.

In advanced X-ray sources, the filament may be used to indirectly heat a secondary electron emitting region that will typically be coated in a dispenser cathode material (e.g. thoriated porous tungsten) with low work function. During operation, the temperature of the dispenser cathode material is generally significantly lower (e.g. 1200K operating temperature) than a standard tungsten filament (>1500K), which means that the thermal power required to heat the dispenser cathode to operating temperature is less than that for the standard tungsten filament and typically in the range 0.3 W-2 W.

This lower operating power is beneficial in terms of reducing overall X-ray tube power dissipation. This is especially important in X-ray sources that have multiple electron guns, such as a multi-focus X-ray tube for use in stationary gantry computed tomography.

In some applications, power dissipation from the electron source(s) can be detrimental to overall system design, especially in applications where there is limited space, where there is limited capacity for thermal heat dissipation, and where multiple electron emitters are required in a single tube envelope, such as in stationary gantry computed tomography systems to be deployed in high throughput baggage screening applications.

Over the past fifty years, considerable work has been conducted on the development of cold cathode emitters—designed to operate at room temperature—which tend to rely on field emission of electrons from sharp points or tips. The latest generation of such emitters tend to take advantage of developments in nano-engineering, such as growth of carbon nanotubes. However, despite significant work, such electron sources still require operation in extreme high vacuum (e.g. 10-9 Torr) and their emission current density is limited by the electric field applied to each emission point and the total surface area of the emission region. In X-ray tube applications, the sensitive point-like emission sources get damaged easily by reverse ion bombardment from ions generated as a result of ablation of the X-ray tube target or other gas molecules present in the vacuum. Therefore, the development of X-ray sources based on field emission has yet to be proven successful.

The limitations of current electron sources for use in low operating power multi-focus X-ray sources is therefore recognized. There is a need for an alternative X-ray source that mitigates the reliability issues seen with field emission based X-ray sources, and reduces operating power substantially compared to standard thermionic electron sources.

### SUMMARY

The following embodiments and aspects thereof are described and illustrated in conjunction with systems, tools and methods, which are meant to be exemplary and illustrative, not limiting in scope.

In some embodiments, the present specification discloses an optical device configured for use in an X-ray inspection system, the optical device comprising: a light source configured to emit light; a photocathode proximate the light source, positioned such that it is in a path of the light emitted by the light source, and configured to emit a first plurality of electrons; a first dynode positioned to receive the first plurality of electrons emitted by the photocathode and configured to emit a second plurality of electrons in response to receiving the first plurality of electrons; and a second dynode positioned to receive the second plurality of electrons emitted by the first dynode and configured to emit a third plurality of electrons in response to receiving the second plurality of electrons.

Optionally, at least a portion of the optical device is enclosed in a vacuum sealed housing and wherein the vacuum sealed housing comprises at least one of glass or metal.

Optionally, the light source is positioned outside the housing.

Optionally, the photocathode comprises a material deposited over an optically transparent glass within the vacuum sealed housing.

Optionally, the optical device further comprises at least one of a grid electrode or a focus electrode.

Optionally, the light source is a light emitting diode (LED).

Optionally, the LED emits at least one of blue light or white light.

Optionally, the light source a LASER.

Optionally, the photocathode, the first dynode, and the second dynode are placed within the vacuum sealed housing.

In some embodiments, the present specification discloses an X-ray inspection system comprising: a stationary X-ray source extending around a scanning volume, wherein the stationary X-ray source comprises: a plurality of source points, wherein each of the plurality of source points is configured to generate X-rays and direct the X-rays into the scanning volume and wherein each of the plurality of source points comprises: a light assembly comprising: a light source configured to emit light; a photocathode proximate the light source, positioned such that it is in a path of the light emitted by the light source, and configured to emit a first plurality of electrons; and a first dynode positioned to receive the first plurality of electrons emitted by the photocathode and configured to emit a second plurality of electrons in response to receiving the first plurality of electrons; and a second dynode positioned to receive the second plurality of electrons emitted by the first dynode and configured to emit a third plurality of electrons in response to receiving the second plurality of electrons; and an anode assembly positioned to receive the third plurality of electrons and configured to convert the third plurality of electrons to said X-rays; an X-ray detector array extending around the scanning volume and arranged to detect X-rays from the anode assembly which have passed through the scanning volume; a conveyor arranged to convey the items through the scanning volume; and at least one processor for processing the detected X-rays to produce images of items passing through the scanning volume.

Optionally, the anode assembly emits the X-rays from a plurality of different emission points.

Optionally, each of the plurality of source points is enclosed in a vacuum sealed housing and wherein the housing comprises at least one of glass or metal.

Optionally, the light assembly further comprises at least one of a grid electrode or a focus electrode.

Optionally, the photocathode comprises a material deposited over an optically transparent glass.

Optionally, the light source is a light emitting diode (LED).

Optionally, the stationary X-ray source comprises the plurality of source points and the anode assembly positioned in an enclosed housing.

Optionally, the LED emits one of blue light or white light.

Optionally, the photocathode and at least one of the first dynode or the second dynode are placed inside a vacuum sealed housing.

In some embodiments, the present specification discloses a method for scanning items using an X-ray inspection system, the method comprising: passing an item to be scanned through an enclosed inspection volume; emitting X-rays from a stationary X-ray source positioned around the inspection volume by: illuminating a photocathode from a light source that emits light towards the photocathode; receiving, at a first dynode, a first plurality of electrons emitted by the photocathode; receiving, at a second dynode, a second plurality of electrons emitted by the first dynode; receiving, at an anode assembly, a third plurality of electrons emitted by the second dynode and converting the third plurality of electrons to said X-rays; detecting X-rays from the stationary X-ray source which have passed through the enclosed inspection volume; and processing the detected X-rays to produce scanning images of the items.

Optionally, a number of the second plurality of electrons is greater than a number of the first plurality of electrons.

Optionally, a number of the third plurality of electrons is greater than a number of the second plurality of electrons.

Optionally, at least one of the photocathode, first dynode, or second dynode is enclosed in a vacuum sealed glass or metal housing.

Optionally, the photocathode is deposited on a surface of a glass positioned inside a vacuum sealed housing.

Optionally, the light source is a light emitting diode (LED).

Optionally, the LED is configured to emit at least one of blue light or white light.

Optionally, each of the steps of illuminating the photocathode, receiving at the first dynode, and receiving at the second dynode are performed within a vacuum.

Optionally, the photocathode receives light from the light source at a first side of the photocathode and emits the first plurality of electrodes from a second side of the photocathode, wherein the first side is positioned opposite the second side.

Optionally, the method further comprises using a third dynode in series with the second dynode.

In some embodiments, the present specification discloses a light assembly for use in an X-ray inspection system for scanning items, the assembly comprising: a light source; and at least two dynodes, wherein a first dynode is positioned to receive electrons emitted by the photocathode and a second dynode is positioned to receive electrons emitted by the first dynode.

Optionally, the assembly is enclosed in a glass or metal envelope.

Optionally, the light source is positioned outside the glass or metal envelope.

Optionally, the assembly further comprises at least one of: a grid and a focus electrode.

Optionally, the light source is a light emitting diode (LED).

Optionally, the LED emits one of blue light or white light.

Optionally, the light source a LASER.

Optionally, the at least two dynodes are placed inside a vacuum housing.

In some embodiments, the present specification discloses a method for scanning items using an X-ray inspection system, the method comprising: emitting X-rays from a stationary X-ray source comprising at least one cathode assembly from which X-rays can be directed through the scanning volume, wherein the emitting from the cathode assembly comprises: emitting light from a light source and directing said light towards a first dynode; generating electrons, using said light, by the first dynode; emitting generated electrons by the first dynode towards a second dynode; and receiving at the second dynode, electrons emitted by the first dynode, wherein the electrons are multiplied and emitted further by the second dynode; converting emitted electrons to X-rays by an anode assembly; detecting X-rays from the anode assembly which have passed through the scanning volume; and processing the detected X-rays to produce scanning images of the items.

Optionally, the cathode assembly is enclosed in a glass or metal envelope.

Optionally, the emitting light from a light source comprises emitting light from a light emitting diode (LED). Optionally, the LED emits one of blue light or white light.

Optionally, the steps of generating electrons by the first dynode; emitting generated electrons by the first dynode towards the second dynode; and receiving at the second dynode, electrons emitted by the first dynode, wherein the electrons are multiplied and emitted further by the second dynode are performed with a vacuum.

Optionally, the method further comprises using at least one more dynode in succession with the second dynode, wherein each dynode multiplies received electrons.

The aforementioned and other embodiments of the present specification shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings:

FIG. 1A is a perspective view of a conventional X-ray inspection system;

FIG. 1B is a schematic diagram illustrating a plurality of views of the scanning unit of FIG. 1A;

FIG. 2A illustrates an X-ray tube cathode assembly, in accordance with some embodiments of the present specification;

FIG. 2B shows a circuit diagram for operation of a photocathode-based electron source shown in FIG. 2A, in accordance with some embodiments of the present specification;

FIG. 3 is a flow chart illustrating some of the exemplary steps of a method for scanning items using an X-ray inspection system, in accordance with embodiments of the present specification;

FIG. 4 is a flow chart illustrating some of the exemplary steps of another method for scanning items using an X-ray inspection system, in accordance with some embodiments of the present specification;

FIG. 5 is a flow chart of a plurality of exemplary steps of a method of manufacturing the X-ray source or electron gun of FIG. 2A; and

FIG. 6 illustrates a variation in photocathode radiation sensitivity at different wavelengths of light.

### DETAILED DESCRIPTION

It is understood by one of ordinary skill in the art that photomultiplier tubes are commonly used in the field of radiation detection to take the flash of light caused by radiation interaction in a scintillation detector (such as sodium iodide, NaI), to convert this to electrons using a photocathode and to then accelerate the generated electrons through an electric field to a first dynode whereby each interacting electron from the photocathode is absorbed and re-emitted in the form of several electrons, each of which is then accelerated towards a second dynode. The process repeats at the second and subsequent dynodes until a large signal has been produced at the final dynode and the resulting amplified signal is recorded at an anode. The whole photomultiplier tube is operated in a standard vacuum, typically in the range 10⁻⁷ to 10⁻⁶ Torr. Typically a gain in the range of n=5 to 20 is achieved at each dynode. For a system with 10 dynodes, this means a gain of n¹⁰ for the photomultiplier as a whole.

Embodiments of the present specification recognize that the photocathode material (for example, Caesium Iodide (CsI)) absorbs optical photons within its bulk which releases an electron into the conduction band through the photoelectric effect. This conduction electron is free to migrate throughout the bulk of the photocathode. If it gets to the surface of the material, it has a good probability of escaping from the photocathode material into the vacuum. Therefore, this is a bulk effect, not a point emission effect, which is controlled by the thickness of the photocathode material, the wavelength of light entering the photocathode and the strength of the electric field at the emission surface of the photocathode. In these respects a photocathode is an ideal source of electrons for an X-ray tube cathode. The emission of the electron source may be therefore switched on and off by modulating the intensity of the applied optical beam at the photocathode. This is a key characteristic of a multi-focus X-ray source in which it is necessary to turn each one of the multiple electron sources within a single X-ray tube on and off with high temporal precision and repeatability.

In embodiments, the present specification provides an inspection system with multiple source points used to scan the scanning volume. In an embodiment, the inspection system is a real-time tomography (RTT) system. In an embodiment, the source points are arranged in a non-circular or substantially rectangular geometry around the scanning volume. In embodiments, the inspection system is cost effective, has a smaller footprint and may be operated using regular line voltage to supply power to the high voltage power supply, which is then used to provide power to the X-ray source.

In various embodiments, the X-ray sources emit fan beams which have different emission points based on the location of the X-ray source points with respect to the imaging volume.

It should be noted that the systems described throughout this specification comprise at least one processor to control the operation of the system and its components. It should further be appreciated that the at least one processor is capable of processing programmatic instructions, has a memory capable of storing programmatic instructions, and employs software comprised of a plurality of programmatic instructions for performing the processes described herein. In one embodiment, the at least one processor is a computing device capable of receiving, executing, and transmitting a plurality of programmatic instructions stored on a volatile or non-volatile computer readable medium.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present specification is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present specification.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

For the purposes of this specification, a filtered back-projection method is defined to describe any transmission or diffraction tomographic technique for the partial or complete reconstruction of an object where a filtered projection is back-projected into the object space; i.e., is propagated back into object space according to an inverse or approximate inverse of the manner in which the beam was originally transmitted or diffracted. The filtered back-projection method is usually implemented in the form of a convolution of filters and directly calculates the image in a single reconstruction step.

For the purposes of this specification an iterative reconstruction method refers to iterative algorithms (versus a single reconstruction algorithm) used to reconstruct 2D and 3D images such as a computed tomography where an image must be reconstructed from projections of an object.

For the purposes of this specification, thermionic emission refers to a thermally induced charge emission process. Thermal energy drives charge carriers over a potential energy barrier, thereby causing an electric current. In conventional X-ray tubes, the charge carriers are electrons emitted from heated cathodes.

For the purposes of this specification, a photocathode is a negatively charged electrode which is coated with a photosensitive compound. The photocathode emits electrons when illuminated, thereby causing an electric current. Photocathodes typically operate in a vacuum.

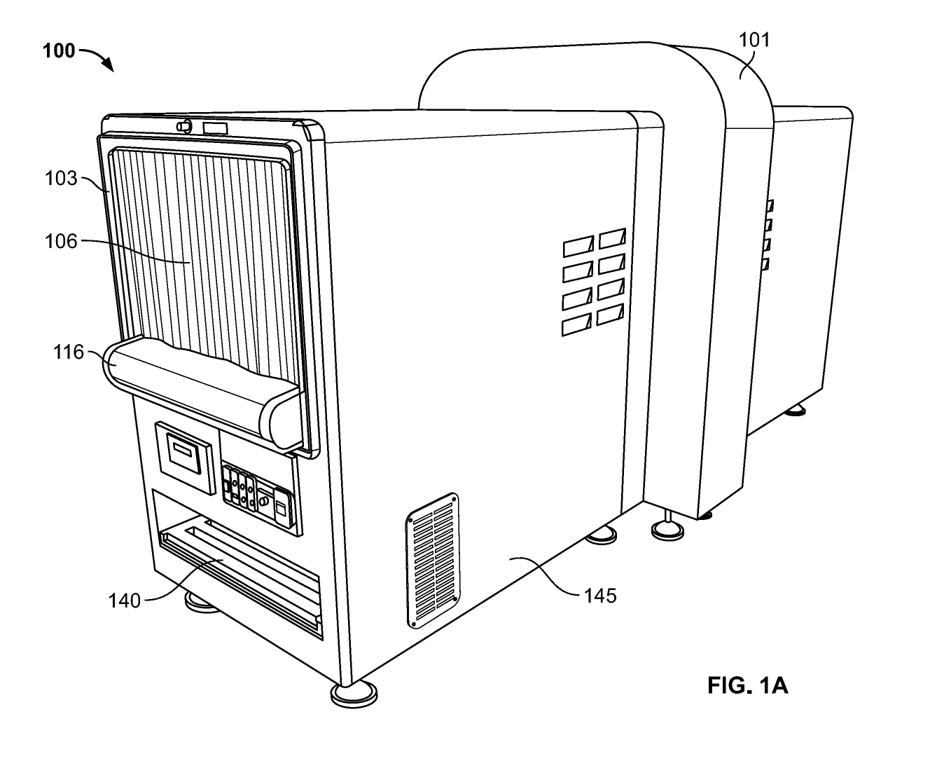

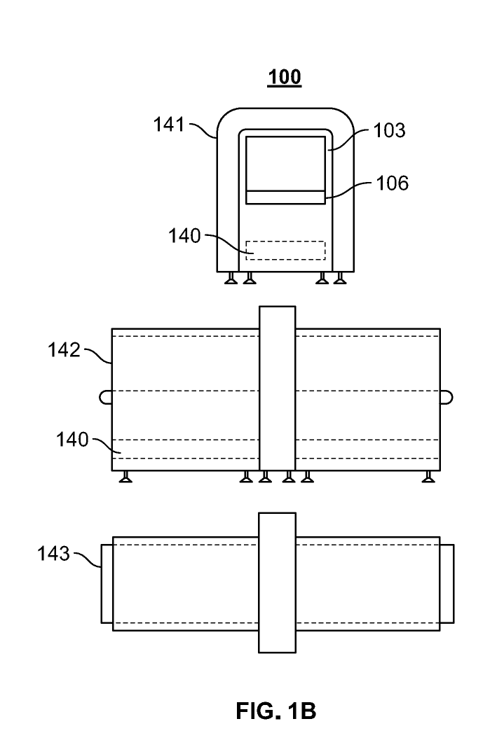

FIG. 1A is a perspective view of a conventional scanning unit 100, shown from a first side 145, comprising a substantially rectangular housing/enclosure 101 for housing a plurality of X-ray source points and detectors. The source points, which are positioned at the surface of an anode, are located in a first plane normal to the plane of a conveyor. The detectors are located in a plane parallel to the source points and are also normal to the plane of a conveyor but offset from the source point plane by a distance ranging from 1 mm to 20 mm. In embodiments, there are between 100 and 1000 source points arranged around the perimeter of the tunnel in a rectangular, circular or other similar shape. It should be appreciated that, in alternate embodiments, the housing 101 may have a circular, or a quadrilateral shape, such as, but not limited to, a square, or any other shape. An object under inspection is conveyed through a first open end or scanning aperture 103, enters the inspection region, and exits through a second open end (opposite to the first open end 103). In accordance with an embodiment, both feed and return conveyor loops pass through a space 116 just below the inspection region 106, while space or compartment 140 is reserved in the base of the scanning system (approximately 200 mm deep) to accommodate automated return of trays when integrated with an automatic tray return handling system.

FIG. 1B illustrates a plurality of views of an exemplary scanning unit 100 of FIG. 1A. The scanning unit 100 may be designed for reduced power usage and reduced noise. Referring now to FIG. 1B, view 141 illustrates a first open end or scanning aperture 103 of the scanning system 100 for objects under inspection to enter the inspection region. In some embodiments, the scanning aperture 103 and thus, inspection volume, has a width of 620 mm and a height of 420 mm. View 142 is a side view (as seen from first side 145 of FIG. 1A) along a longitudinal direction of the scanning unit 100. View 143 is a top view along the longitudinal direction of scanning unit 100. It should be noted that the longitudinal length of scanning system 100 as shown in view 143 is to accommodate for higher levels of X-ray scatter from the object under inspection which is caused by the higher beam current that is necessarily used to produce a clear image. Views 141, 142 also illustrate the space 140 through which tray can pass when integrated with an automatic tray return handling system.

While FIGS. 1A and 1B illustrate exemplary scanning systems for implementing the various embodiments of the present specification, other scanning systems may also incorporate these embodiments. In embodiments, the present application relates to U.S. Pat. No. 8,085,897 entitled “X-Ray Scanning System”, and issued on Dec. 27, 2011, and its family members. The present application also relates to the scanning system described in U.S. Pat. No. 7,876,879, entitled “X-Ray Tomography Inspection Systems”, and issued on Jan. 25, 2011, and its family members. Embodiments of the electron source of the present specification may also be applicable to the X-ray tube described in U.S. Pat. No. 8,824,637, entitled “X-Ray Tubes”, and issued on Sep. 2, 2014. Embodiments of the electron source of the present specification may also be applicable to the electron gun described in U.S. Pat. No. 9,618,648, entitled “X-Ray Scanners”, and issued on Apr. 11, 2017. Embodiments of the electron source of the present specification may also be applicable to the X-ray scanner described in U.S. Pat. No. 8,243,876, entitled “X-ray Scanners”, and issued on Aug. 14, 2012. Embodiments of the electron source of the present specification may also be applicable to the X-ray scanner described in U.S. Pat. No. 7,949,101, entitled “X-Ray Scanners and X-Ray Sources Therefor”, and issued on May 24, 2011. All of the above-mentioned applications are herein incorporated by reference.

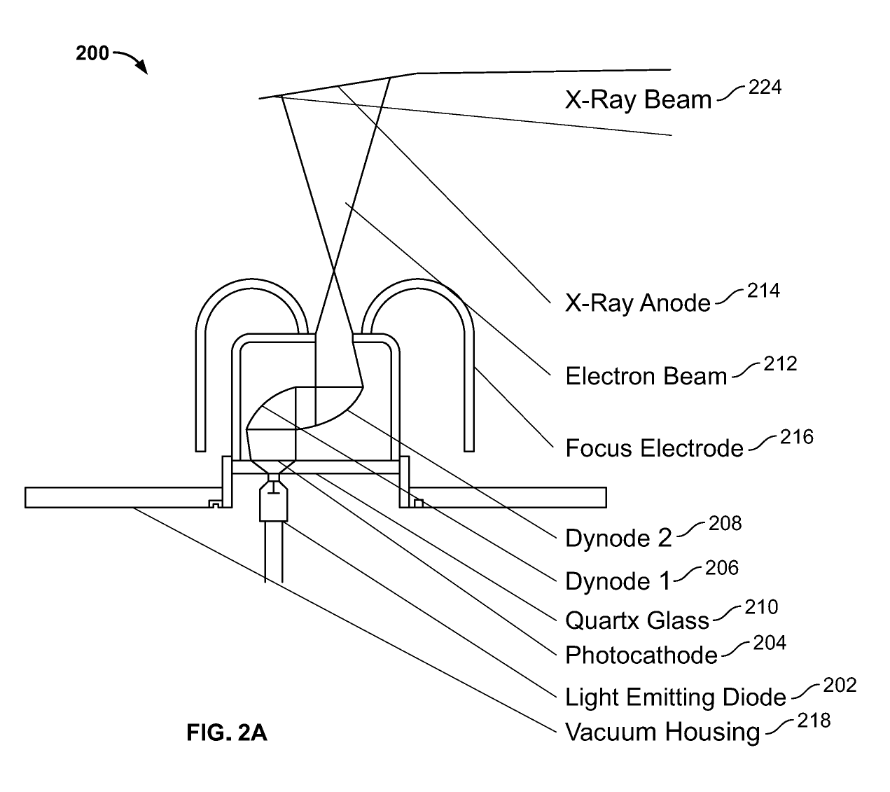

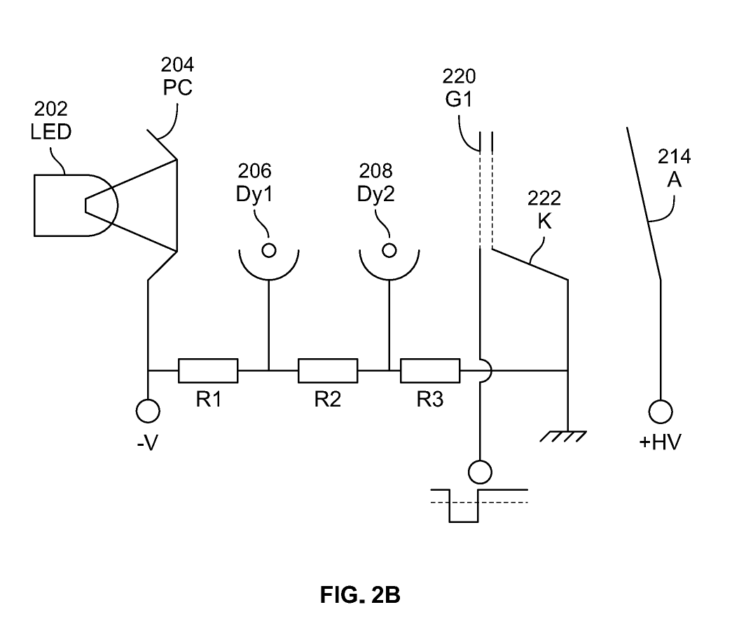

FIG. 2A illustrates an X-ray tube cathode assembly 200, in accordance with some embodiments of the present specification. In embodiments, assembly 200 is placed inside a vacuum housing 218, and comprises an optically transparent window through which a beam of light is passed from a light source. In embodiments, the wavelength of emission of diode 202 is matched with the wavelength of maximum emissivity of a photocathode material. The wavelength of light emitted by the light source such as diode 202 is chosen such that optical photons have sufficient energy to excite a valence electron into the conduction band of the photocathode material. In embodiments, this may mean operation in the blue to green wavelength range depending on materials chosen. In one embodiment, the light source is a light emitting diode (LED) 202, which, optionally, has a high brightness. In accordance with embodiments of the present specification, brightness is defined in terms of power (Watts (W)) or luminosity (milliCandela (mCd)). A high brightness LED has power in the range of 0.5 W to 2 W with luminosity in the range of 500 mCd to 8000 mCd depending on the frequency (for example, typically blue wavelengths will have lower luminosity than green wavelengths). In embodiments, the brightness and luminosity are dependent upon many factors, including but not limited to required beam current, photocathode material, and wavelength of excitation so it should be noted that these parameters may be adjusted depending on the requirements of the overall system of the present specification.

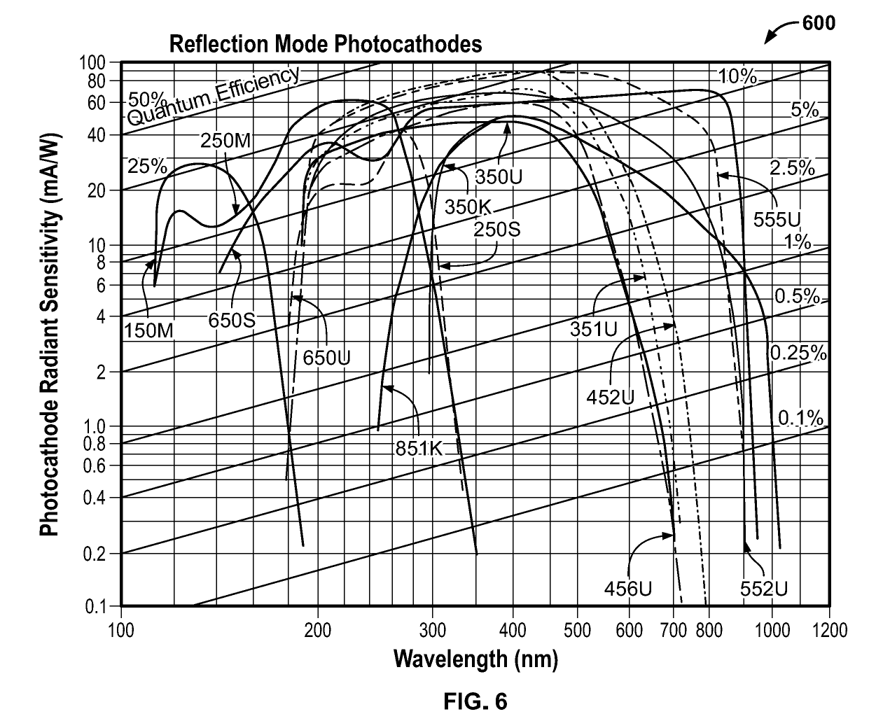

In embodiments, the photocathode material chosen is at least one of CsI, CsSb, and GaAsP. FIG. 6 illustrates a graph 600 showing a variation in photocathode radiation sensitivity at different wavelengths of light. In embodiments, less than 0.2 mA is required from the photocathode, which may be obtained from a 10 mW LED brightness source, assuming 100% conversion efficiency from power to light and 100% Quantum Efficiency (QE). In reality, the QE of the photocathode is around 10%, which implies that only one in ten arriving optical photons create a photoelectron from the photocathode. Therefore, in embodiments, an optical power ranging from 50 mW to 300 mW is used for LED brightness in the present specification. In an embodiment, an optical power of 100 mW is used for the LED brightness in the present specification. The brightness of diode 202 is chosen such that the diode is capable of delivering the requisite photocathode current. In another embodiment, the light source is a LASER, or any other light source permissible by the implementations of the present specification, which provides sufficient brightness to operate a photocathode. In embodiments, the optically transparent window is fitted with optically transparent glass in the wavelength range of interest (typically a silica or quartz glass 210). The glass is selected because it is transparent and efficient at transmitting white light or blue light. The glass 210 is also used to close or seal the vacuum envelope.

In embodiments, the photocathode is placed in proximity to diode 202. In some embodiments, the photocathode is placed within a range of 0.1 mm to 5 mm from diode 202. Diode 202 is used to excite one photocathode. In an embodiment of a multi-focus X-ray tube comprising multiple photocathodes, each photocathode is associated with one photodiode, similar to diode 202. In some embodiments, electron sources arrays are built in multiples of eight (8) or sixteen (16) in which case, there are 8 or 16 individual LED's irradiating 8 or 16 individual photocathode/dynode assemblies. Light emitting diode 202 is used to commence or “kick-off” a cascade of electron generation. Light emitting diode 202 operates at a ground potential and creates a pulsed light output. In embodiments, each light emitting diode 202 operates outside of the vacuum envelope so that they can be easily replaced. This optical beam generated by each light emitting diode 202 is absorbed by a photocathode 204 which is held at a fixed electrical potential by a transparent metal film (for example indium tin oxide, ITO). In embodiments, photocathode 204 has properties including, but not limited to, the ability to provide efficient light absorption; optimal conversion of optical energy to emitted electrons (good emissivity); stability under high light irradiation conditions; and a low work function.

In an embodiment, the material employed for photocathode 204 is evaporated onto glass 210 when heated under vacuum. In one embodiment, photocathode 204 is heated when the tube is baked during subsequent manufacturing using a vacuum oven. Photocathode material 204 may be placed on the vacuum side of glass 210 as a small drop or pellet. In other words, photocathode 204 coats glass 210 on the side of the glass that is opposite to the side facing light emitting diode 202 (the vacuum side of glass 210).

In embodiments, distance between first dynode 206 and photocathode 204 ranges from 2 mm to 50 mm. In one embodiment, a first dynode 206 is placed at a distance of 5 mm from photocathode 204. First dynode 206, placed in the vacuum across photocathode 204, is at a negative potential, however, its negative potential is less than that of photocathode 204. The angle between photocathode 204 and first dynode 206 is engineered to provide a relatively uniform field across the surface of photocathode 204 and first dynode 206. In some embodiments, a potential difference of 30V to 400V, and preferably 50V to 200V, is maintained between photocathode 204 and first dynode 206 and between subsequent dynodes. Dynode 206, and any other dynode are shaped liked arcs, in accordance with some embodiments of the present specification. The shape of the dynodes is designed to obtain a relatively uniform electric field at the surface of each dynode to achieve a stable gain from each dynode. The dynode shape is also designed to create an electric field from the photocathode to first dynode 206 and then again from first dynode 206 to the second dynode 208 and the next dynode and so forth. In some other embodiments, each dynode has any other shape that is able to perform the functions prescribed in the embodiments of this specification. Electrons emitted from photocathode 204 are accelerated in an electric field to a first dynode 206 which multiplies each arriving electron by a factor of typically 5 to 20. The multiplication factor depends on several factors including dynode material and the energy of the arriving electrons. The angle between first dynode 206 and a second dynode 208, and similarly between each consecutive dynode, is engineered to provide relatively uniform field across their surfaces. In embodiments, the angle between first dynode 206 and second dynode 208 ranges from 0 degrees (measured from the bottom left of the first dynode 206 to the bottom left of the second dynode 208) to 90 degrees (measured from the top right of the first dynode 206 to the bottom left of the second dynode 208). In embodiments, the dynodes are all positioned in a single plane. In one embodiment, each dynode, including dynode 206, is coated with a simple metal or a low electron affinity coating such as Cesium Antimony or an antimony-tin alloy. In an embodiment, the photocathode metal that is employed may be characterized by a low vacuum work function.

Electrons emitted from first dynode 206 are accelerated to second dynode 208 where a further multiplication occurs. In embodiments, distance between first dynode 206 and second dynode 208 ranges from 2 mm to 20 mm. Second dynode 208, placed in the vacuum across first dynode 206, is at a negative potential, however, its negative potential is less than that of first dynode 206. In embodiments, arriving electron energy at dynode 208 is approximately in the range of 50 eV to 200 eV. In embodiments, the arriving energy is proportional to the accelerating voltage, which may be in a range of 30V to 400V, and preferably 50V to 200V. The energy of the arriving electrons is driven by the applied potential difference and the distance between the various parts of the dynode surfaces. The electrons that hit dynode 208 are further multiplied by a factor of 5 to 20. As a result, there is a multiplicative gain of electrons. Overall, a gain of 25 to 400 is achieved through the two dynode stages depending on photocathode 204 and the optimization, orientation, geometry, and applied voltages of first dynode 206 and second dynode 208. The preferred configuration of the photocathodes and dynodes relative to one another is such that the electric field is equalized over the surface of dynodes 206 and 208 and photocathode 204 as far as possible since the probability of an electron escaping into the vacuum between dynodes is driven by the electric field at the dynode surface, which, in turn, depends on the dynode geometry.

In some embodiments, more than two dynodes are deployed to increase the multiplicative gain of electrons. The multiplicative gain stages result in decoupling the stage of electron creation from the process of electron generation for forming an X-ray beam. As a result, the first source of electrons, in this case photocathode 204 is operated with a light source enabling lower temperatures for operating the cathode.

In some embodiments, the photocathode 204 may be removed and the light from the external light source may irradiate the first dynode directly in order to generate secondary electron emission.

In some embodiments, electrons emitted in an electron beam 212 from second dynode 208 are extracted into an electron focusing structure to direct the generated electrons of beam 212 to an X-ray anode 214. In embodiments, the electron focusing structure is focus electrode or grid electrode to shape the electric field around the electron emitter for controlling the beam cross-section so that a suitable focal spot is formed on the anode target. In some embodiments, the focus electrode is formed from a refractory material, such as tungsten or molybdenum, in order to survive high energy ion bombardment of ions liberated from residual gas atoms or atoms ablated from the target. The focus electrode may be open, or may be in a mesh form with multiple apertures, including a simple cruciform grid.

In embodiments, a focusing electrode is an electrode to which a potential is applied to control the cross-sectional area of the electron beam in an X-ray tube. In some embodiments, the focusing optic(s) is in a cylindrical form which focuses the electron beam from the final dynode in both lateral and longitudinal (X-Y) directions at once. This approach may be suited to a compact electron source design with similar X-Y dimensions at the electron emitter, such as a button source. In some alternative embodiments, separate linear focusing structures are used to focus independently in X and Y directions. The independent focusing approach may be suited to electron sources which are extended in one direction compared to the other, such as a line source. In embodiments, an electron focusing structure 216 and any electrostatic grid (not shown) that is placed above second dynode 208 is held at ground potential to act as the primary point of discharge for any high voltage breakdown that may occur during tube conditioning as part of tube manufacture or at any point thereafter during system operation.

As shown in FIG. 2A, dynodes 206, 208 may be configured to introduce a spatial offset between photocathode 204 and the point of emission of electron beam 212. In embodiments, the spatial offset may be defined as the distance (in the horizontal direction) between the vertical axes that pass through centers of each dynode 206 and 208. This offset ensures that any reverse ion bombardment affects electron focusing structure 216 and final dynode only and does not affect photocathode 204 or first dynode 206. This helps to ensure long tube lifetime even in relatively poor vacuum operation.

In embodiments, an electron gun operates at a beam current ranging from 2 mA to 50 mA, and preferably ranging from 4 mA to 20 mA. In one embodiment, the electron gun operates at 4 mA, preferably for small tunnel checkpoint applications. In one embodiment, the electron gun is used to operate at 20 mA, preferably for large tunnel hold baggage screening applications. A beam current of 20 mA equates to generation of approximately 10¹⁵ electrons per second at the surface of photocathode 204, or photocathode current of 800 μA, given dynode gains of 5. This photocathode current reduces to 50 μA for dynode gain of 20. These currents are achieved using off-the-shelf, low cost, high brightness, options for light emitting diode 202.

FIG. 2B shows a circuit diagram for operation of a photocathode 204-based electron source shown in FIG. 2A, in accordance with some embodiments of the present specification. In embodiments, photocathode (PC) 204 is illuminated by light emitting diode (LED) 202. Electrons from PC 204 are firstly accelerated to Dynode 1 (Dy1) 206 and then to Dynode 2 (Dy2) 208. The dynodes from Dy2 208 then accelerate towards an optional perforated grid (G1) 420 and then to a final cathode (K) 222. Electrons that pass from cathode 222 into the main X-ray vacuum envelope then accelerate to X-ray anode (A) 214.

In some embodiments, grid G1 220 is introduced to cathode assembly 200. The effect of G1 220 is to create a controlled field region between G1 220 and cathode, K 222. This allows beam current to be modulated by adjusting the potential on G1 220 relative to K 222. Switching G1 220 to a positive potential with respect to K 222 switches the electron beam off completely regardless of the illumination of LED 202. Thus, G1 220 provides a second level of control of the emitted electron beam, including a wider on-to-off beam current ratio plus controllable beam current by setting the potential of G1 220 relative to K 222. In some embodiments, one grid is provided for each emitter in order to eliminate cross-talk.

In an alternative embodiment, assembly 200 is constructed without grid G1 220. In this embodiment, electron emission is controlled solely by the switching of LED 202. Referring simultaneously to FIGS. 2A and 2B, when LED 202 is switched on and illuminating photocathode 204, an electron beam 212 is produced and this is accelerated into the main vacuum envelope to produce an X-ray beam 224. When LED 202 is switched off, no photocathode electrons are generated and no X-ray beam is produced. Therefore, X-ray emission is controlled directly by modelling the brightness of LED 202. Intensity of LED 202 is directly proportional to the X-ray tube beam current.

In one embodiment, multiple photocathodes, dynodes and cathodes are connected to a single set of potentials that are supplied to the electron source components via a single set of electrical vacuum feedthroughs, one set of feedthroughs per X-ray tube. This is very efficient compared to thermionic sources where multiple electrical feedthroughs are required to provide power to the filaments and to control the respective grid signals. The present embodiments do not include any thermionic element and so the electron source operates at zero power other than when actively generating an X-ray beam.

In an alternative embodiment, a field emission source is used for electron generation. In an embodiment, the field emission source and focusing optics similar to the configuration described in context of FIG. 2B are used for electron generation. Cathode and grid materials for a cathode in a field emission source may employ a highly refractory material such as tungsten or molybdenum. The electric field (E) formed is due to an electrical potential (V) applied between the grid and cathode and the distance (d) between them (E=V/d). In embodiments, the field emission source is placed in a closer proximity to the grid, relative to the proximity of LED 202 and grid G1 220.

Embodiments of cathode assembly 200 can be implemented in various embodiments of an X-ray inspection system. Cathode assembly 200 may be a component of an X-ray source point, where multiple X-ray source points form an X-ray screening system. Alternatively, a single source point can be used in a standard single focus X-ray source. Alternatively, two source points can be used in a dual focus source to create broad (high power) and fine (low power) focus, for example. Embodiments of the present specification provide an advantage over conventional cathode assemblies as the cathode temperatures of the present embodiments can be kept at room temperature, whereas heated cathodes in conventional cathode assemblies operate at above 1500K.

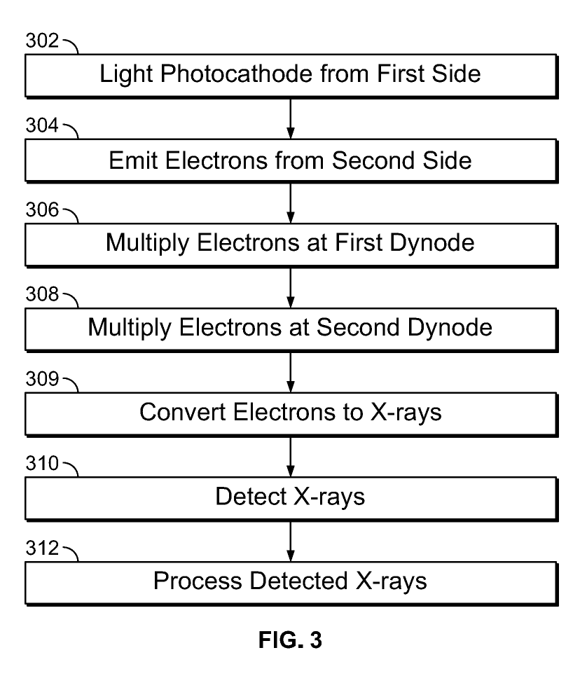

FIG. 3 is a flow chart illustrating some of the exemplary steps of a method for scanning items using an X-ray inspection system, in accordance with embodiments of the present specification. In embodiments, a stationary X-ray source is formed by multiple X-ray source points that surround a scanning volume that may be of arbitrary shape (generally rectangular or circular). The X-ray source points are positioned at the surface of anode are located in a first plane normal to the plane of a conveyor while the detectors are located in a plane parallel to the source points and are also normal to the conveyor but offset from the source point plane by a distance that ranges from 1 mm to 20 mm. In embodiments, there are between 100 and 1000 source points arranged around the perimeter of the tunnel in a rectangular, circular or other similar shape. Each source point includes a cathode assembly, such as the one described in context of FIGS. 2A and 2B. At 302, a photocathode is lit from a first side using a light source. In embodiments, the light source is an LED that emits white light. In embodiments, the light source is an LED that emits blue light. It should be noted herein that the responsivity of the photocathode is generally over a broad spectral range and that white LEDs are generally brighter than blue LEDs, although either may be employed. At 304, lighting the photocathode causes the photocathode to emit electrons from a second side, which is opposite to the first side. In embodiments, the photocathode is positioned or deposited over an optically transparent glass. At 306, a first dynode receives the electrons emitted by the photocathode. The first dynode multiplies received electrons and emits them towards a second dynode. At 308, the second dynode receives the electrons from the first dynode and further multiplies them and emits the multiplied electrons. In some embodiments, the system includes at least one or more additional dynodes in succession after the second dynode. Each dynode is configured to receive electrons from the previous dynode and further multiply electrons. In an alternate embodiment, the light source irradiates the first dynode directly without the use of a separate photocathode and electrons emitted from the first dynode are directed to a second and subsequent dynode(s). In yet another alternative embodiment, electrons are generated with a field electron emission source instead of a light source. In embodiments, steps from 304 to 308 are performed in a vacuum. In embodiments, a glass or metal enclosure is used to perform these steps. At step 309, an anode assembly receives the electron generated by the dynodes and converts them to X-rays. Subsequently, at 310, the X-rays from the anode assembly that have passed through the scanning volume are detected by one or more detectors. At 312, the detected X-rays are processed by a processor in order to produce scanning images of the items being scanned.

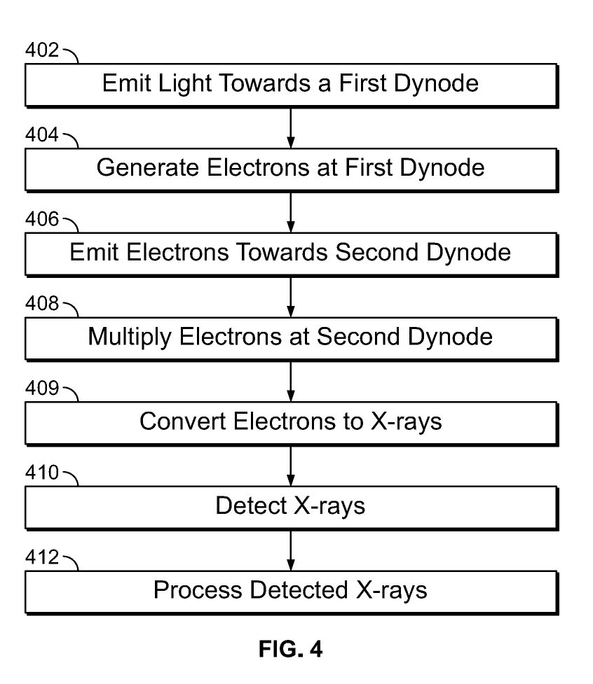

FIG. 4 is a flow chart illustrating some of the exemplary steps of another method for scanning items, in accordance with some embodiments of the present specification. In an embodiment, as described with respect to FIG. 4, a light source irradiates the first dynode directly without the use of a separate photocathode. Electrons emitted from the first dynode are directed to a second dynode and optionally, subsequent dynodes. At step 402, a light source emits light towards a first dynode. Each source point includes a light source assembly. In embodiments, the light source is an LED, or a LASER. At step 404, the first dynode receives the light from the light source and thereby generates electrons. At step 406, the first dynode emits the generated electrons and directs them towards a second dynode. At 408, the second dynode receives the electrons from the first dynode, multiplies them and subsequently, emits the multiplied electrons. In some optional embodiments, the system includes at least one or more additional dynodes in succession after the second dynode. Each dynode is configured to receive electrons from the previous dynode and further multiply electrons. In some embodiments, the light source assembly is configured similar to the system illustrated in the context of FIGS. 2A and 2B, without the photocathode. In embodiments, steps 404 through 408 are performed in a vacuum. In embodiments, a glass or metal enclosure is used to create the vacuum. At step 409, an anode assembly converts the electrons generated by the dynodes to X-rays. Subsequently, at step 410, the X-rays from the anode assembly that are transmitted through the scanning volume are detected by one or more detectors. At step 412, the detected X-rays are processed by a processor in order to produce images of the items being scanned.

Referring back to FIG. 1A, it should be appreciated that in contrast to conventional rotating gantry systems, the firing pattern for the multi-focus X-ray source 102 is not constrained to move in a standard helical rotation about a baggage under inspection. Thus, in various embodiments, the source firing pattern may be fixed or random with uniform or non-uniform dwell time at each source point 120. In various embodiments, the dwell time ranges from 50 μs to 500 μs per scan projection. In some embodiments, the dwell time is 200 μs per scan projection.

In various embodiments, in order to determine substantially accurate measures for Z-Effective and Density in the reconstructed RTT images, both sinogram data (the multi-energy “raw” data produced by the X-ray detectors for each source projection) and the reconstructed image data from one or more multi-energy bins is used in determining threat type for each object segmented from the 3D image data. In embodiments, the reconstructed image is available as soon as the trailing edge of a conveyor tray leaves the RTT imaging region of the scanning unit 100.

In accordance with some embodiments, the scanning unit 100 is configured to achieve reconstructed image voxels of 0.8 mm×0.8 mm×0.8 mm over an inspection tunnel size of 620 mm wide×420 mm. This is equivalent to a slice image size of 775 pixels (width)×525 pixels (height). For a conveyor tray length of 0.8 m, there will be 1,000 slices in each 3D image. In some embodiments, the RTT system spatial resolution is 1.0 mm at the center of the inspection tunnel. In embodiments, the RTT system is configured to achieve Z-effective resolution of +/−0.2 atomic numbers with density resolution at the center of the inspection tunnel of +/−0.5%.

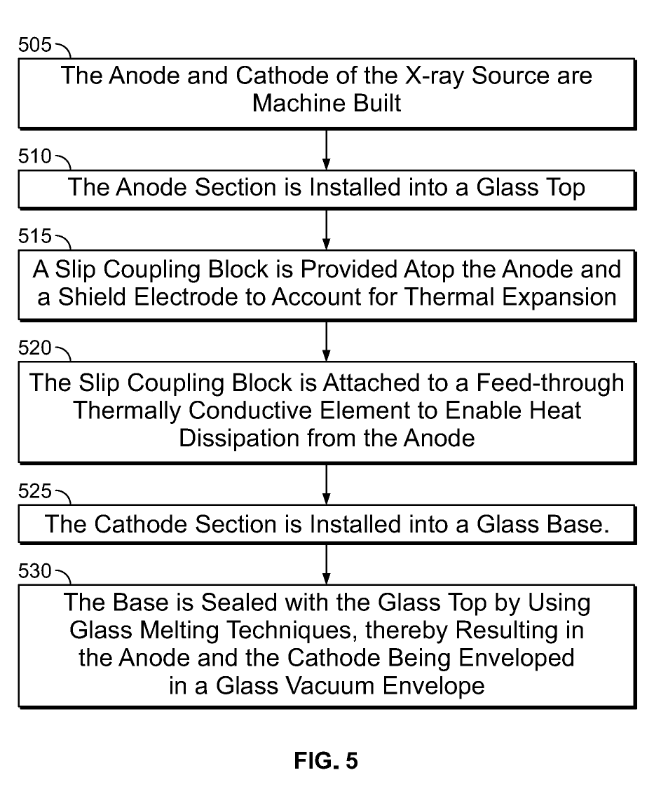

FIG. 5 is a flow chart of a plurality of exemplary steps of a method of manufacturing the X-ray source or electron gun of FIG. 2A. At step 505, the anode and cathode of the X-ray source are machine built. At step 510, the anode section is installed into a glass or metal top. At step 515, a slip coupling block is provided atop the anode and a shield electrode to account for thermal expansion. At step 520, the slip coupling block is attached to a feed-through thermally conductive element to enable heat dissipation from the anode. Next, at step 525, the cathode section is installed into a glass or metal base. Finally, at step 530, the base is sealed with the glass or metal top by using glass melting or metal welding techniques, thereby resulting in the anode and the cathode being enveloped in a glass or metal vacuum envelope.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. An optical device configured for use in an X-ray inspection system, the optical device comprising:
a light source configured to emit light;
a photocathode proximate the light source, positioned such that it is in a path of the light emitted by the light source, and configured to emit a first plurality of electrons;
a first dynode positioned to receive the first plurality of electrons emitted by the photocathode and configured to emit a second plurality of electrons in response to receiving the first plurality of electrons; and
a second dynode positioned to receive the second plurality of electrons emitted by the first dynode and configured to emit a third plurality of electrons in response to receiving the second plurality of electrons.

2. The optical device of claim 1 wherein at least a portion of the optical device is enclosed in a vacuum sealed housing and wherein the vacuum sealed housing comprises at least one of glass or metal.

3. The optical device of claim 2 wherein the light source is positioned outside the housing.

4. The optical device of claim 2, wherein the photocathode comprises a material deposited over an optically transparent glass within the vacuum sealed housing.

5. The optical device of claim 1 wherein the optical device further comprises at least one of a grid electrode or a focus electrode.

6. The optical device of claim 1 wherein the light source is a light emitting diode (LED).

7. The optical device of claim 6 wherein the LED emits at least one of blue light or white light.

8. The optical device of claim 1 wherein the light source a LASER.

9. The optical device of claim 2 wherein the photocathode, the first dynode, and the second dynode are placed within the vacuum sealed housing.

10. An X-ray inspection system comprising:
a stationary X-ray source extending around a scanning volume, wherein the stationary X-ray source comprises:
a plurality of source points, wherein each of the plurality of source points is configured to generate X-rays and direct the X-rays into the scanning volume and wherein each of the plurality of source points comprises:
a light assembly comprising:
a light source configured to emit light;
a photocathode proximate the light source, positioned such that it is in a path of the light emitted by the light source, and configured to emit a first plurality of electrons;
a first dynode positioned to receive the first plurality of electrons emitted by the photocathode and configured to emit a second plurality of electrons in response to receiving the first plurality of electrons; and
a second dynode positioned to receive the second plurality of electrons emitted by the first dynode and configured to emit a third plurality of electrons in response to receiving the second plurality of electrons; and
an anode assembly positioned to receive the third plurality of electrons and configured to convert the third plurality of electrons to said X-rays;
an X-ray detector array extending around the scanning volume and arranged to detect X-rays from the anode assembly which have passed through the scanning volume;
a conveyor arranged to convey the items through the scanning volume; and
at least one processor for processing the detected X-rays to produce images of items passing through the scanning volume.

11. The X-ray inspection system of claim 10 wherein the anode assembly emits the X-rays from a plurality of different emission points.

12. The X-ray inspection system of claim 10 wherein each of the plurality of source points is enclosed in a vacuum sealed housing and wherein the housing comprises at least one of glass or metal.

13. The X-ray inspection system of claim 10 wherein the light assembly further comprises at least one of a grid electrode or a focus electrode.

14. The X-ray inspection system of claim 10 wherein the photocathode comprises a material deposited over an optically transparent glass.

15. The X-ray inspection system of claim 10 wherein the light source is a light emitting diode (LED).

16. The X-ray inspection system of claim 10 wherein the stationary X-ray source comprises the plurality of source points and the anode assembly positioned in an enclosed housing.

17. The X-ray inspection system of claim 15 wherein the LED emits one of blue light or white light.

18. The X-ray inspection system of claim 10 wherein the photocathode and at least one of the first dynode or the second dynode are placed inside a vacuum sealed housing.

19. A method for scanning items using an X-ray inspection system, the method comprising:
passing an item to be scanned through an enclosed inspection volume;
emitting X-rays from a stationary X-ray source positioned around the inspection volume by:
illuminating a photocathode from a light source that emits light towards the photocathode;
receiving, at a first dynode, a first plurality of electrons emitted by the photocathode;
receiving, at a second dynode, a second plurality of electrons emitted by the first dynode;
receiving, at an anode assembly, a third plurality of electrons emitted by the second dynode and converting the third plurality of electrons to said X-rays;
detecting X-rays from the stationary X-ray source which have passed through the enclosed inspection volume; and
processing the detected X-rays to produce scanning images of the items.

20. The method of claim 19, wherein a number of the second plurality of electrons is greater than a number of the first plurality of electrons.

21. The method of claim 20, wherein a number of the third plurality of electrons is greater than a number of the second plurality of electrons.

22. The method of claim 19 wherein at least one of the photocathode, first dynode, or second dynode is enclosed in a vacuum sealed glass or metal housing.

23. The method of claim 19 wherein the photocathode is deposited on a surface of a glass positioned inside a vacuum sealed housing.

24. The method of claim 19 wherein the light source is a light emitting diode (LED).

25. The method of claim 24 wherein the LED is configured to emit at least one of blue light or white light.

26. The method of claim 19 wherein each of the illuminating the photocathode, receiving at the first dynode, and receiving at the second dynode are performed within a vacuum.

27. The method of claim 19 wherein the photocathode receives light from the light source at a first side of the photocathode and emits the first plurality of electrodes from a second side of the photocathode, wherein the first side is positioned opposite the second side.

28. The method of claim 19 further comprising using a third dynode in series with the second dynode.
